(19) United States
(12) Patent Application Publication
(10) Pub. No.: US 2018/0007932 A1
(43) Pub. Date: Jan. 11, 2018

(54) METHOD FOR FRACTIONATING COMPONENTS OF A BIOMASS OF PROTEIN-RICH MICROALGAE

(71) Applicant: Roquette Freres, Lestrem (FR)

(72) Inventor: Samuel Patinier, Quesnoy Sur Deule (FR)

(73) Assignee: Roquette Freres, Lestrem (FR)

(21) Appl. No.: 15/546,236

(22) PCT Filed: Jan. 25, 2016
(86) PCT No.: PCT/FR2016/050138

§ 371 Date: Jul. 25, 2017

(30) Foreign Application Priority Data
Jan. 26, 2015 (FR) 15 50571

(51) Int. Cl. A23J 1/00; C07K 14/405; A23J 3/34; C07K 1/36; C12N 1/12; C12P 21/00

(52) U.S. Cl. CPC A23J 1/009; A23L 33/18; A23L 27/235; C07K 14/405; A23L 27/88; A23J 3/347; C12N 1/12; C12P 21/00; C07K 1/36

(57) ABSTRACT

The invention relates to a method for fractionating the components of a biomass of protein-rich microalgae of the genus Chlorella, characterized in that it comprises the following steps:
providing a microalgal biomass produced by fermentation,
optionally, washing the biomass so as to eliminate the interstitial soluble compounds,
thermal permeabilization of the biomass at a temperature of between 50 and 150° C., preferably 100 and 150° C., for a duration of between 10 seconds and 5 minutes, preferably for a duration of between 5 seconds and 1 minute,
separation between the biomass thus permeabilized and the soluble fraction by a centrifugation technique, more particularly multistage centrifugation,
optionally, recovery and clarification of the soluble fraction obtained in this way by microfiltration so as to remove residual insoluble substances therefrom,
separation of the preceding soluble fraction by precipitation, so as to obtain a peptide isolate and a peptide concentrate.

The present invention relates to a method for fractionating components of the biomass of protein-rich microalgae.

### PRESENTATION OF THE PRIOR ART

It is well known to those skilled in the art that chlorellae are a potential source of food, since they are rich in proteins and other essential nutrients.

They are described as containing 45% protein, 20% fat, 20% carbohydrate, 5% fiber and 10% minerals and vitamins.

Given their abundance and their amino acid profile, microalgal proteins are thus considered as an alternative source to soy or pea proteins in food.

The protein fraction may also be exploited as a functional agent in the cosmetic, or even pharmaceutical, industries.

However, developments in food applications for microalgal proteins have not been significant, since the presence in said fractions of undesirable compounds (such as chlorophyll) leads to undesired changes in color, flavor and structure of the food compositions containing them.

To increase their potential in food applications and also to increase their commercial value, these proteins must therefore be extracted from the microalgae without affecting their molecular structure.

“Soft” extraction techniques would therefore be necessary to isolate proteins with high solubilities and good technical and functional properties, but the rigidity of microalgal cell walls, especially of green microalgae, is fundamentally in contradiction to this, since it disrupts the extraction and integrity of the intracellular proteins.

Thus, on the contrary, conventionally “hard” physical or chemical conditions are employed to break the microalgal cell wall.

Numerous studies thus propose technologies of alkaline dissolution type, extraction by organic solvent type or high-pressure homogenization type.

In these technological choices, the denaturing of proteins was not however considered to be bothersome, since most of these methods were developed for purposes of analyses or intended to provide a substrate for the enzymatic digestion producing protein hydrolyzates.

However, an effective disintegration method preserving the intergrity of the cell components should maximize not only the yield, but also the quality of the products extracted.

In other words, a method for optimized disintegration of the wall must for example avoid:
chemical contamination of the targeted products,
using a breaking energy which is too high; the latter possibly causing irreversible denaturation or degradation of the intracellular molecules of interest.

Moreover, for large-scale productions, it is important for the process chosen to be transposable to this scale.

Finally, the introduction of this cell disintegration step must be easy and must not have a negative impact on the subsequent method/treatment steps.

All these limitations influence the efficiency of the disintegration method and by the same token its energy consumption.

This is why the bead mill technology is preferred, since it is considered to be efficient for releasing intracellular proteins in their native form.

In a bead mill, the cells are agitated in suspension with small spherical particles. The breaking of the cells is caused by the shear forces, the milling between the beads, and the collisions with beads.

The description of an appropriate bead mill is, for example, given in the patent U.S. Pat. No. 5,330,913. These beads break the cells so as to release the cell content therefrom. A suspension of particles of smaller size than the cells of origin is then obtained in the form of an “oil-in-water” emulsion.

This emulsion is generally atomized and the water is eliminated, leaving a dry powder containing, however, a heterogeneous mixture composed of cell debris, interstitial soluble compounds, and oil.

The difficulty to be solved in the use of these cell disintegration technologies is the isolation of solely the intracellular content (to the exclusion of the membrane debris, sugars, fibers and fats) and the preservation, especially, of the quality of the protein load.

In the case of the microalga of the genus Tetraselmis sp, Anja Schwenzfeier et al (Bioresource Technology, 2011, 102, 9121-9127) proposed a method guaranteeing the solubility and the quality of the aminogram of the proteins isolated and with contaminants (such as coloring substances) removed, comprising the following steps:
cell disintegration by bead mill,
centrifugation of the milled microalgal suspension,
dialysis of the supernatant,
passage over ion-exchange resin,
dialysis of the eluate,
decolorizing, then,
washing and resuspending.

However, this laboratory method (for treating 24 g of biomass) cannot be scaled up to an industrial scale, where the bead mill method is rather used to recover a complete biomass.

Alternative solutions have been proposed, completely changing the technology for releasing the intracellular content of the microalgae, such as pulsed-field electrical treatment.

This is because exposure of biological cells to a high-intensity pulsed electric field can modify the structure of the cell membrane.

The external field causes charging of the membrane. At a sufficient transmembrane voltage (0.5-1 V), the molecular arrangement of the phospholipids changes, which results in the membrane losing its barrier role, making it permeable. Depending on the conditions used, this membrane permeabilization can be reversible or irreversible.

For efficient extraction of the intracellular compounds, those skilled in the art using this technology remain, however, advised to bring about an irreversible permeabilization of the membrane, thereby resulting in its disintegration.

This rupture of the membrane then facilitates the release of the cell content and, in the case of the use of a supplementary solvent-extraction technique, also facilitates the penetration of the solvent into the cell.

This technique, although promising, can unfortunately not be extrapolated to an industrial scale for treating a biomass produced in a reactor of 1 to 200 m³.

As a result, there remains an unmet need to provide a technology for weakening microalgal cell walls that is capable of releasing the intracellular content without disintegrating the cell or impairing the quality of the components thereof.

### SUBJECT OF THE INVENTION

The Applicant company has found that this need can be met by combining a method for the thermal permeabilization of microalgal cells with steps of centrifugation and precipitation by modifying the properties of the medium.

The Applicant company thus goes against a technical prejudice which says that thermal methods for cell disruption, just like the shear forces caused by mechanical disintegration, are technologies that are instead used for degrading or denaturing the products originating from microalgae (Richmond, 1986, Handbook of Microalgal Mass Culture. CRC Press, Inc—Molina Grima et al., 2003, Recovery of microalgal biomass and metabolites: process options and economics, Biotechnol. Adv. 20:491-515).

Moreover, once released from the intracellular compartment, the recovery of the peptide isolate is performed easily, given that the heat treatment developed by the Applicant company does not lead to disintegration of the cell wall.

Finally, the method of the invention makes it possible above all to recover and upgrade the residual biomass, and also the coproducts of the peptide isolate.

### DETAILED DESCRIPTION OF THE INVENTION

The present invention thus relates to a method for fractionating components of the biomass of protein-rich microalgae:
by performing a method of thermal permeabilization of the cell membrane, followed by
recovery and upgrading of the residual biomass thus permeabilized and of its coproducts.

More precisely, the method according to the invention is a method for fractionating the components of a biomass of protein-rich microalgae of the genus Chlorella, characterized in that it comprises the following steps:
providing a microalgal biomass produced by fermentation,
optionally, washing the biomass so as to eliminate the interstitial soluble compounds,
thermal permeabilization of the biomass at a temperature of between 50 and 150° C., preferably between about 80 and 150° C., for a time of between about 10 seconds and about 5 minutes, preferably for a time of between about 5 seconds and about 1 minute,
separation between the biomass thus permeabilized and the soluble fraction by a centrifugation technique, more particularly multistage centrifugation,
optionally, recovery and clarification of the soluble fraction obtained in this way by microfiltration so as to remove residual insoluble substances therefrom,
purification of the preceding soluble fraction by precipitation, so as to obtain a peptide isolate and a peptide concentrate.

The term “approximately” is intended to mean a value range comprising plus or minus 10% of the indicated value, preferably plus or minus 5% thereof. For example, “approximately 10” means between 9 and 11, preferably between 9.5 and 10.5.

Choice of the Microalga

Preferably, the microalgae of the Chlorella genus are chosen from the group consisting of Chlorella vulgaris, Chlorella sorokiniana and Chlorella protothecoides, and are more particularly Chlorella protothecoides.

In one particular embodiment, the strain is Chlorella protothecoides (strain UTEX 250—The Culture Collection of Algae at the University of Texas at Austin—USA). In another embodiment, the strain is the strain CCAP211/8D—The Culture Collection of Algae and Protozoa, Scotland, UK).

Choice of the Fermentation Conditions

The culturing under heterotrophic conditions and in the absence of light conventionally results in the production of a chlorella biomass having a protein content (evaluated by measuring the nitrogen content N×6.25) of 45% to 70% by weight of dry cells.

It is preferred to start with a biomass of protein-enriched microalgae having, for example, a protein content, expressed as N.6.25, of greater than 60%. In this case, the Applicant company recommends using a novel method which it has developed, and which comprises:
a first fermentation step, limited in nitrogen, in which the pH regulation is performed with an NH₃/KOH mixture, and then
a second step of removal of this nitrogen limitation by a pH regulation performed with NH₃ alone.

These operating conditions thus make it possible rapidly to obtain a biomass with a protein content of greater than 60% of N.6.25, of the order of 65% of N.6.25, and low coloration. The yield is from 45 to 50% by weight of solids, and the final concentration of biomass is between 80 and 120 g/l.

Treatment of the Biomass

The biomass is then collected by solid-liquid separation, by frontal or tangential filtration or by any means known, moreover, to those skilled in the art.

Optionally, the Applicant company then recommends washing the biomass in such a way as to eliminate the interstitial soluble compounds by a succession of concentration (by centrifugation)/dilution of the biomass.

For the purposes of the invention, the term “interstitial soluble compounds” means all the soluble organic contaminants of the fermentation medium, for example the water-soluble compounds such as the salts, the residual glucose, the oligosaccharides with a degree of polymerization (or DP) of 2 or 3, or the peptides.

This biomass purified in this way of its interstitial soluble compounds is then preferentially adjusted to a solids content of between 15 and 30% by weight, preferably to a solids content of between 20 and 30%.

Thermal Permeabilization of the Biomass

The heat treatment is performed at a temperature of between 50 and 150° C., preferably between about 80 and 150° C., for a time of between about 10 seconds and about 5 minutes, preferably for a time of between about 5 seconds and about 5 minutes, preferably for a time of between about 10 seconds and about 1 minute. In a preferred embodiment, the heat treatment is performed at a temperature of about 140° C. for about 10 seconds. In another preferred embodiment, the heat treatment is performed at a temperature of about 85° C. for about 1 minute.

This treatment makes it possible to allow the intracellular components to diffuse into the reaction medium.

Finally, at the end of these steps, the biomass is cooled preferably to a temperature of below 40° C., or even refrigerated at about 4° C.

Without wishing to be bound by a particular theory, the Applicant company considers that the thermal treatment, performed under these operating conditions, could thus act as a membrane weakening process which allows the spontaneous release of the soluble components of the intracellular compartment, or even of the extracellular matrix.

In addition to the ionic substances, organic substances such as carbohydrates (predominantly DP1 and DP2), the peptides and the polypeptides are drained out of the cell.

Conversely, the lipids and hydrophobic organic compounds remain in the cells, thereby clearly demonstrating that the cells are permeabilized and not lyzed or destroyed.

The method according to the invention does not therefore result in the formation of an emulsion, but indeed of an aqueous suspension.

The release of all these soluble substances through the permeabilized membrane is similar to a process of free diffusion of dialysis type.

Consequently, a lag time may be necessary in order to allow sufficient diffusion after the heat treatment which permeabilizes the membrane.

In the literature, the process for pulsed-field permeabilization of yeast membranes in order to extract the proteins therefrom requires from 4 h to 6 h (Ganeva et al., 2003, Analytical Biochemistry, 315, 77-84).

According to the invention, a much shorter reaction time is used, of between about 5 seconds and about 5 minutes.

Separation of the Permeabilized Biomass and of the Soluble Fraction

Separation is then performed between the biomass thus permeabilized and the soluble fraction by a centrifugation technique, more particularly multistage centrifugation.

If necessary, the soluble fraction thus obtained may be clarified by microfiltration so as to free it of the residual insoluble matter and, depending on its solids content, a concentration by evaporation or by any other means additionally known to those skilled in the art may be performed before the purification that follows.

The resulting soluble fraction is finally essentially composed of protein (50-80% w/w) and carbohydrates (5-25% w/w).

Upgrading of the Residual Biomass

The residual biomass, from which the soluble matter has been separated, may undergo upgrading as a whole ingredient whose nutritional profile is recalibrated.

Specifically, the protein content is reduced—since it is partly entrained in the form of peptides in the soluble matter—and this reequilibrates the balance in favor of the carbohydrate and lipid fraction.

The residual biomass after separation by centrifugation may be “also milled” (according to the desired applicative properties), preferentially by mechanical milling.

Conventionally, the biomass is stabilized (pH readjusted (about 7), addition of antioxidants, etc.) and is then heat-treated (pasteurization for the purpose of bacteriological control) before drying by atomization. A step of concentration by evaporation may precede the heat treatment (optimization coupled with drying).

Purification of the Protein Isolate by Precipitation

The method of the invention leads here to the isolation of peptides of interest, by precipitation by modifying the properties of the medium.

The Applicant company thus recommends proceeding as follows:
promoting the precipitation of all or part of the peptide fraction by modifying the physicochemical properties of the medium.
The cooling of the crude soluble matter, obtained as described in the preceding steps, triggers a phenomenon of precipitation of part of the soluble peptides.
It is observed that the precipitation is rather selective toward the higher molecular weights. The cooling temperature is below 10° C., preferably below 4° C.
Certain operating conditions make it possible to promote this phenomenon: besides the temperature, the pH must be between 2.5 and 6.5 and preferably close to the pHi, i.e. between 3 and 5.
Similarly, the ionic strength of the medium may be adapted to promote precipitation.
Thus, by greatly reducing the ionic strength, the phenomenon of “salting-in” may be attenuated, and the solubility of the proteins may thus be reduced (by reducing the solvation layer).
Thus, a demineralization operation prior to the precipitation may be added. This is performed on cationic and anionic resins, dialysis, filtration or by any means additionally known to those skilled in the art.
Conversely, by greatly increasing the ionic strength, the available water decreases via the phenomenon of “salting-out”, and in this way the proteins have a tendency to precipitate. This method is not preferred since pronounced demineralization would then be necessary on the protein isolate thus extracted.
   In the same perspective of modulating the solvation layer, the polarity of the medium may be reduced (with dehydration of the medium) by adding a solvent such as ethanol which will make it possible to generate more quantitative precipitation of the protein fraction by greatly reducing its solubility.
by recovering the precipitated fraction which is then optionally concentrated before drying.
Separation of the precipitated fraction is performed by simple decantation and recovery of the heavy phase or optionally by centrifugation under optimum temperature conditions.
The pH may optionally be readjusted before drying.
Drying is performed by atomization, lyophilization or by any other means additionally known to those skilled in the art.
Prior to drying, the incorporation of a step of concentration by evaporation may make it possible to optimize the operation in energy terms. It may especially be justified if a solvent such as ethanol is used to perform its recycling.

Exploiting these approaches makes it possible to purify a fraction with a high content of peptides and polypeptides from the residual salts and sugars.

A soluble protein isolate is then obtained at greater than 90% by weight.

Upgrading of the Residue

When the isolate has thus been extracted, the soluble phase (light phase after separation) may be upgraded as such as protein concentrate (depending on its residual protein content) or may undergo a new purification process to extract therefrom the residual peptides.

This may especially be justified depending on the experimental conditions when the precipitation is partial (e.g. partial precipitation in aqueous phase). In this case, the residual peptides, which are generally of lower molecular weight (more soluble) may be extracted by modifying the physicochemical environment in the same way as described for the protein isolate.

For example, the incorporation of a solvent such as ethanol may be performed at this stage to generate precipitation of this residual protein fraction by greatly decreasing its solubility.

The action of the solvent will be all the more efficient if the residue is dehydrated beforehand. This may be performed up to a certain solids content by evaporation or up to complete drying (for example by atomization).

After precipitation, the pH of this fraction may optionally be readjusted, and concentration by evaporation (which may allow recycling of the solvent) is then optionally performed before drying by atomization, lyophilization or by any means additionally known to those skilled in the art.

The invention will be understood more clearly from the following examples which are intended to be illustrative and nonlimiting.

### EXAMPLES

### Example 1

### Production of Chlorella protothecoides with a High Protein Content

The strain used is a Chlorella protothecoides (strain CCAP211/8D—The Culture Collection of Algae and Protozoa, Scotland, UK).

Preculture:
150 mL of medium in a 500 mL conical flask;
Composition of the medium: 40 g/L of glucose+10 g/L of yeast extract.

Incubation is performed under the following conditions:
time: 72 h;
temperature: 28° C.;
shaking: 110 rpm (Infors Multitron Incubator).

Culturing in Batch and Then Fed Batch Mode

Preparation and Initial Batch Medium
prepare and filter a mixture of KOH at 400 g/l (41%)/NH3 at 20% v/v (59%);
sterilize 20 L fermenter at 121° C./20 min;
inoculate with 2 conical flasks of 500 mL of preculture (OD_{600 nm} of 15);
brought to pH 4.5 with 20% v/v NH₃;
starting shaking speed of 300 rpm;
aeration: 20 L/min of air;
pO₂ regulation at 30%;

Feed
glucose: 500 g/L
ammonium sulfate: 5 g/L
diammonium phosphate: 20 g/L
phosphoric acid: 16 g/L
magnesium sulfate heptahydrate: 12 g/L
iron sulfate: 170 mg/L
calcium nitrate: 610 mg/L
solution of trace elements: 45 mL/L
solution of vitamins: 4.5 mL/L

It is important to note that the feedstock of ammonium salts, magnesium salts and phosphoric acid was developed so as to limit the salt content of the fermentation medium and was optimized so as to maintain the N.6.25 content of the final decolorized biomass.

Solution of trace elements

| Ingredients | (g/l) |
|---|---|
| CuSO₄ | 0.22 |
| ZnSO₄ | 7 |
| MnSO₄ | 4 |
| Citric acid | 30 |

Solution of vitamins

| Ingredients | (g/l) |
|---|---|
| Thiamine HCl | 2.25 |
| Biotin | 0.11 |
| Pyridoxine | 1.1 |

Fermentation Procedure
provide the equivalent of 20 g/L before inoculation
when the glucose concentration is 0 g/L, start the feed in exponential profile (μ=0.07 h⁻¹);
regulation of the pH at 5.2 with the 41% KOH/59% NH₃ mixture
when 2 kg of glucose have been consumed by the microalga, switch the system to pH regulation with NH₃ alone.

Results:

This fermentation procedure makes it possible to obtain a biomass with more than 65% protein, expressed as N.6.25.

### Example 2

### Thermal Permeabilization of the Chlorella protothecoides Biomass and Recovery of the Soluble Fraction by Precipitation

The biomass produced according to Example 1 is harvested at a cell solids content of 105 g/L with a purity of 80% (purity defined by the ratio of the solids content of the biomass to the total solids content).

It is then:
washed and concentrated by inline dilution [1:1] (V_{water}Vₘᵤₛₜ) and centrifuged on an Alfa Laval FEUX 510 plate centrifuge and brought to a solids content of 220 g/L and to a purity of 93% (purity defined by the ratio of the solids content of the biomass to the total solids content), and then
the pH is adjusted to 7 with potassium hydroxide,
heat treatment with HTST on an indirect steam plate heat exchanger bringing the biomass to 85° C. maintained for 1 minute by holding, followed by cooling to 4° C. on a glycol-water plate heat exchanger.

The heat treatment is performed at a moderate scale so as to limit the partial dissolution of the biomass, the purity of which decreases to 68%.

By definition, the salting-out of the soluble matter in the extracellular medium leads to a decrease in the fraction of cell solids relative to the total solids content.

At this stage, the composition of the biomass is as follows:
total amino acids: 48.73%
total sugars: 27.02%
total fatty acids: 15.10%
ash and others: 9.15%

Separation of the Crude Soluble Matter

Separation of the soluble matter derived from the salting-out by thermal permeabilization of biomass is performed by centrifugal separation.

In order to optimize the separation yield and quality, a slight dilution [0.5:1] (V_{water}Vₘᵤₛₜ) is performed inline on the second stage (on a configuration with two Alfa Laval FEUX 510 centrifuges in series) with recycling of the supernatant from the second stage into the first. The supernatant from the first stage is thus recovered and the clarified soluble matter is concentrated.

This “crude” soluble matter has the following composition:
total amino acids: 77.3%
total sugars: 17.6%
ash and others: 5.1%

Purification of the Protein Isolate

A sample of soluble matter taken after separation is used for a purification directed toward obtaining the protein isolate.

In order to selectively precipitate the peptide fraction, 750 g of crude soluble matter with a solids content of 9.5% are placed in a jacketed reactor with stirring.

The pH of the crude soluble matter is adjusted to 4.5 with phosphoric acid.

After stopping the stirring, the temperature is lowered to 4° C.

These conditions are maintained for 8 hours.

Decantation of the heavy phase enriched in peptides of higher molecular weight is thus performed.

The heavy phase is then extracted by simple phase separation in a separating funnel, with a mass yield of 28% and has a solids content of 37.2%.

This extract is lyophilized to a solids content of 97%.

The composition of this isolate is detailed below:
total amino acids: 95.9%
total sugars: 2.44%
ash and others: 1.66%

The amino acid profile distribution of the protein isolate is as follows:
glutamic acid: 49.9%
arginine: 47.21%
others: 2.89%

The isolate is thus characterized by a richness of the order of 95% of amino acids formed essentially by arginine and glutamic acid (on the basis of the distribution analysis of the total amino acids).

Purification of the Residue

The light phase, after precipitation and separation of the isolate, may undergo a purification so as to concentrate the protein fraction that has not precipitated (of lower molecular weight).

After separation (with a mass yield of 72%), this phase, initially with a solids content of 8.9%, is concentrated by evaporation (15 mbar, −43° C. on a Buchi R-215 laboratory rotavapor) to a solids content of 45.4% so as to partially dehydrate the medium in order subsequently to promote the action of the ethanol.

At this stage, the concentrate has the following composition:
total amino acids: 68.5%
total sugars: 23.46%
ash and others: 8.04%

In order to precipitate the protein fraction, dehydration by addition of ethanol is performed.

A volume of ethanol (per volume of concentrate) is added, and protein aggregation resulting from the loss of solubility in the medium takes place virtually instantaneously.

The pellet is recovered by centrifugation at 4000 g for 10 minutes (Beckman Coulter Avanti J-20 XP).

It is then dried to a solids content of 92.3% in a vacuum oven for 24 hours.

The composition of the extract thus obtained is detailed below:
total amino acids: 73.19%
total sugars: 20.45%
ash and others: 6.36%

This extract may then be upgraded as a protein concentrate.

### Example 3

### Treatment of the Residual Biomass after Dissolution

The protein-rich crude insoluble matter obtained in Example 2 is separated from the residual biomass, which may be treated with a process allowing it to be upgraded.

The extracted biomass, at a cell solids content of 22%, is milled on a horizontal bead mill module (Netzsch LME 500-0.6 mm zirconium silicate beads) to a degree of milling of 85%.

The milled cellular material is then adjusted to pH 7 with 50% potassium hydroxide.

Concentration on an SPX forced-circulation evaporator is performed by continuous feeding of a loop in which the temperature is adjusted to 75° C. before entry of the flash under vacuum with the temperature maintained at 40° C. in which the evaporation takes place.

The concentrated biomass is continuously withdrawn from the flash toward the SPX UHT module to perform a heat treatment with preheating at 70° C. followed by direct injection of steam on a scale of about 10 seconds at 140° C. and flash cooling to 40° C. under vacuum.

The biomass is then atomized to a solids content of 95% on a GEA Filtermat FMD 200 atomizer.

The biomass thus obtained has the following composition:
total amino acids: 27.1%
total fatty acids: 27.1%
total sugars: 35.8%
ash and others: 10%

The biomass thus obtained has the advantage of having an equilibrated nutritional profile in the carbohydrate, protein and lipid fraction. As presented below, the amino acid profile is moreover reequilibrated by selective upstream removal of the soluble fraction rich in arginine and glutamic acid.

The amino acid distribution in the biomass is as follows:
aspartic acid: 6.05
threonine: 3.91%
serine: 3.56%
glutamic acid: 23.84%
glycine: 3.56%
alanine: 5.69%
valine: 4.27%
isoleucine: 2.31%
leucine: 5.87%
tyrosine: 2.49%
phenylalanine: 3.20%
lysine: 3.74%
histidine: 1.60%
arginine: 25.98%
proline: 3.91%

1. A method for fractionating components of a biomass of protein-rich microalgae of genus Chlorella, comprising:
providing a microalgal biomass produced by fermentation,
optionally, washing the biomass so as to eliminate interstitial soluble compounds,
thermal permeabilization of the biomass at a temperature of between 50 and 150° C., for a time of between about 10 seconds and 5 minutes,
separation between the biomass thus permeabilized and a soluble fraction by a centrifugation technique,
optionally, recovery and clarification of the soluble fraction by microfiltration so as to remove residual insoluble substances therefrom, and
purification of the preceding soluble fraction by precipitation, so as to obtain a peptide isolate and a peptide concentrate.

2. The method according to claim 1, wherein, the microalgae of Chlorella genus are chosen from the group consisting of Chlorella vulgaris, Chlorella sorokiniana and Chlorella protothecoides.

3. The method according to claim 1, wherein the biomass of protein-rich microalgae is prepared by a method which comprises:
a first fermentation step, deficient in nitrogen, in which pH regulation is performed with an NH₃/KOH mixture, and then
a second step of removal of this nitrogen deficiency by a pH regulation performed with NH₃ alone.

4. The method according to claim 1, wherein the heat treatment is at a temperature of between about 80 and 150° C., for a time of between about 5 seconds and about 5 minutes.

5. The method according to claim 1, wherein the heat treatment is performed at a temperature of about 85° C. for about 1 minute or at a temperature of about 140° C. for about 10 seconds.

6. The method according to claim 1, wherein the permeabilized biomass is subsequently treated by:
milling, preferentially mechanical milling,
stabilization at pH 7,
pasteurization,
atomization.

7. The method according to claim 1, wherein the purification of the soluble fraction is performed by
precipitation at a cooling temperature of less than 10° C., optionally with adjustment of the pH to a value of between 2.5 and 6.5,
centrifugation or decantation, so as to obtain a heavy phase corresponding to the peptide isolate, and a light phase, and
concentration and drying of the peptide isolate.

8. The method according to claim 1, wherein the residual peptides are extracted from the light phase by precipitation with ethanol.
